(19) United States
(12) Patent Application Publication
(10) Pub. No.: US 2015/0337251 A1
(43) Pub. Date: Nov. 26, 2015

(54) MICROFLUIDIC CHIP FOR CULTURING MICROORGANISMS AND METHOD OF OPERATING THE SAME

(71) Applicant: National Tsing Hua University, Hsinchu City (TW)

(72) Inventors: Ya-Tang Yang, Hsinchu City (TW); Chih-Chung Chiang, Hsinchu City (TW); Sze-Bi Hsu, Hsinchu City (TW)

(21) Appl. No.: 14/552,977

(22) Filed: Nov. 25, 2014

(30) Foreign Application Priority Data
May 26, 2014 (TW) 103118219

(51) Int. Cl. C12M 3/06; C12Q 1/18; C12M 1/02; C12M 1/00; C12M 1/34

(52) U.S. Cl. CPC C12M 23/16; C12M 23/34; C12M 29/00; C12M 41/00; C12M 27/00; C12Q 1/18

(57) ABSTRACT

The present invention provides a microfluidic chip for culturing microorganism and a method of operating the same. The microfluidic chip includes a first input unit, a second input unit, a connection unit connected to the first and second input units, a plurality of control valves, and a ring-shaped storage structure connected to the connection unit and having first and second growth chambers that store a microbial solution. The first and second input units provide first and second solutions, respectively. The first solution is transferred into the first or second growth chamber through the connection unit to treat and discharge a portion of the microbial solution. The second solution is transferred into the first or second growth chamber to discharge the first solution. The control valves are actuated to mix the second solution and remainder of the microbial solution in the ring-shaped storage structure.

### BACKGROUND OF THE INVENTION

1. Field of the Invention

This invention relates to microfluidic chips, and, more particularly, to a microfluidic chip for culturing microorganisms and a method of operating the microfluidic chip.

2. Description of Related Art

In 1949, Jacques Monod wrote, “The study of the growth of bacterial culture does not constitute a specialized subject or a branch of research; it is the basic method of microbiology.” Traditionally, the two most common methodologies for bacteria culture have been serial transfer dilution or batch growth mode and the chemostat. In batch growth mode, the microbial population is allowed to grow exponentially in a closed environment test tube) with a fixed amount of nutrient medium. Once a growth cycle is finished, a fraction of the microbe is removed and a fresh medium is inoculated to permit another growth cycle. In the chemostat mode, the microbial population is diluted continually with fresh medium to maintain the nearly steady microbial population. Traditional culture with liquid broth and agar plates are both reagent and labour consuming and efforts have been made to develop automated miniaturized bioreactors with working volumes from the micro- and nanoliter scales down to the scale of single cells. These microfluidic-based chemostat devices have been demonstrated to measure the growth rate under different condition and follow lineages with the resolution down to single cell level. For example, Groisman's flow based microchemostat still been widely used, for example, to monitor the response under the influence of antibiotic drug due to its simplicity in design. It is argued that the nanoliter is the mesoscopic scale for microbiology as the nanometer is for physics and electronics. At this scale there is a sufficiently large microbial population to give a statistically meaningful growth rate, which is important for antibiotic drug resistance measurement. At the nanoliter scale, surface effects are sufficiently pronounced that biofilm formation will play a major role in microbial growth. It is believed that more than 80 percent of clinical infections are caused by biofilms. The biofilm has been studied extensively using microfluidic devices. The vast majority of the study uses flow cell devices for seeding the biofilm.

Microorganisms culturing techniques are important in biological research and biological industry. However, a microorganism culturing device according to the prior art is bulky and expensive. A nutrient solution for culturing microorganisms is scarce in the art and very expensive. Accordingly, the microorganisms thus produced are also expensive. Besides, a growth amount of the microorganisms and reactions of the microorganisms with drugs (e.g., antibiotics) are difficult to be controlled. In recent years, a microfluidic chip of a small size comes to the market, hoping to reduce the manufacture cost and the cost of the nutrient solution.

A microfluidic chemostat proposed by U.S. Pat. No. 8,426,159 comprises a growth chamber having a plurality of partitions, each of which is isolated from the others by one or more actuatable valves. The microfluidic chemostat further comprises a supply circuit that supplies a nutrient solution into the growth chamber. U.S. Pat. No. 8,426,159 further proposed a 2×3 array of microfluidic chip. Therefore, a plurality of chemostatic experiments can be conducted in a parallel manner.

However, the microfluidic chip of the prior art has a complicated structure and a high cost.

Therefore, how to overcome the drawbacks of the prior art is becoming an important issue in the art.

### SUMMARY OF THE INVENTION

The present invention provides a microfluidic chip for culturing microorganisms, comprising: a first input unit that provides a first solution; a second input unit that provides a second solution; a connection unit connected to the first input unit and the second input unit; a ring-shaped storage structure connected to the connection unit and having a first growth chamber and a second growth chamber that store a microbial solution; and a plurality of control valves disposed at the first growth chamber, wherein the first solution is transferred through the connection unit into the first growth chamber or the second growth chamber to treat and discharge a portion of the microbial solution, the second solution is transferred through the connection unit into the first growth chamber or the second growth chamber to discharge the first solution, and the control valves, after actuated, mix the second solution and remainder of the microbial solution in the ring-shaped storage structure.

In an embodiment, the microfluidic chip further comprises a first valve disposed between the first input unit and the connection unit, a first control unit connected to the first valve, a second valve disposed between the second input unit and the connection unit, and a second control unit connected to the second valve.

In an embodiment, the microfluidic chip further comprises a mixing unit connected to the control valves to actuate the control valves and mix the second solution and the remainder of the microbial solution in the ring-shaped storage structure according to a predetermined rotation direction.

In an embodiment, the microfluidic chip further comprises at least one third input unit, at least one third valve, and at least one third control unit, wherein the third input unit provides at least one third solution, the third valve is disposed between the third input unit and the connection unit, and the third control unit is connected to the third valve.

The present invention further provides a method of operating a microfluidic chip for culturing microorganisms, the method comprising: providing a microfluidic chip that has a first input unit, a second input unit, a connection unit, a ring-shaped storage structure, and a plurality of control valves, wherein the connection unit is connected to the first input unit and the second input unit, the ring-shaped storage structure is connected to the connection unit, the ring-shaped storage structure has a first growth chamber and a second growth chamber that store a microbial solution, and the control valves are disposed at the first growth chamber; inputting a first solution from the first input unit and transferring the first solution through the connection unit into the first growth chamber or the second growth chamber to treat and discharge a portion of the microbial solution; inputting a second solution from the second input unit and transferring the second solution through the connection unit into the first growth chamber or the second growth chamber to discharge the first solution; and actuating the control valves to mix the second solution and remainder of the microbial solution in the ring-shaped storage structure.

In an embodiment, the method further comprises controlling a first valve by a first control unit of the microfluidic chip to input the first solution from the first input unit; and controlling a second valve by a second control unit of the microfluidic chip to input the second solution from the second input unit.

In an embodiment, the method further comprises actuating the control valves by a mixing unit of the microfluidic chip to mix the second solution and the remainder of the microbial solution in the ring-shaped storage structure according to a predetermined rotation direction.

In an embodiment, the method further comprises providing at least one third solution by at least one third input unit of the microfluidic chip; and controlling at least one third valve by at least one third control unit of the microfluidic chip to input the third solution from the third input unit.

In an embodiment, the first solution is a lysis solution or a cleaning solution, the second solution is a nutrient solution or a buffer solution, and the third solution is an antibiotic solution.

In a microfluidic chip for culturing microorganisms and a method of operating the same according to the present invention, a first input unit, a second input unit and a third input unit provide a first solution (e.g., a lysis solution), a second solution (e.g., a nutrient solution) and a third solution (e.g., an antibiotic solution), respectively, and transfer the first to third solutions to the first or the second growth chamber of the ring-shaped storage structure, to lysis a biological membrane, mix (dilute) the microbial solution and control the growth of microorganisms.

Therefore, the present invention removes a biological membrane on the pipe wall of the ring-shaped storage structure, and dilutes the microbial solution such that the microbial solution has a concentration equal to (½)^{N} of its original concentration after n periods. Therefore, a growth amount of the microorganisms is controlled to be within a predetermined range and stable, and the drug reactions of the microorganisms with antibiotics can also be obtained.

A microfluidic chip according to the present invention has a nano-level size, a micro capacity and a micro volume, and consumes less nutrient solution. Compared with the prior art, the microfluidic chip according to the present invention has a more simplified structure and a lower cost.

The present invention can sustain the biofilm growth with a cyclic growing planktonic population as the cell supply and can also be tuned for different extent of the biofilm growth simply by changing the plumbing protocol.

### BRIEF DESCRIPTION OF DRAWINGS

The invention can be more fully understood by reading the following detailed description of the preferred embodiments, with reference made to the accompanying drawings, wherein:

FIG. 1 is a top view of a microfluidic chip for culturing microorganisms according to the present invention;

FIGS. 2A-2G are top views illustrating a method for operating a microfluidic chip for culturing microorganisms according to the present invention;

FIG. 3 is an image of a biological membrane adhered to a pipe wall of a ring-shaped storage structure according to the present invention;

FIG. 4A is a growth curve of an amount of micro microorganisms obtained by a microfluidic chip for culturing microorganisms and a method of operating the microfluidic chip according to the present invention; and

FIG. 4B is another growth curve of an amount of micro microorganisms obtained by a microfluidic chip for culturing microorganisms and a method of operating the microfluidic chip according to the present invention.

### DETAILED DESCRIPTION OF THE INVENTION

The following illustrative embodiments are provided to illustrate the disclosure of the present invention, these and other advantages and effects can be apparently understood by those in the art after reading the disclosure of this specification. The present invention can also be performed or applied by other different embodiments. The details of the specification may be on the basis of different points and applications, and numerous modifications and variations can be devised without departing from the spirit of the present invention.

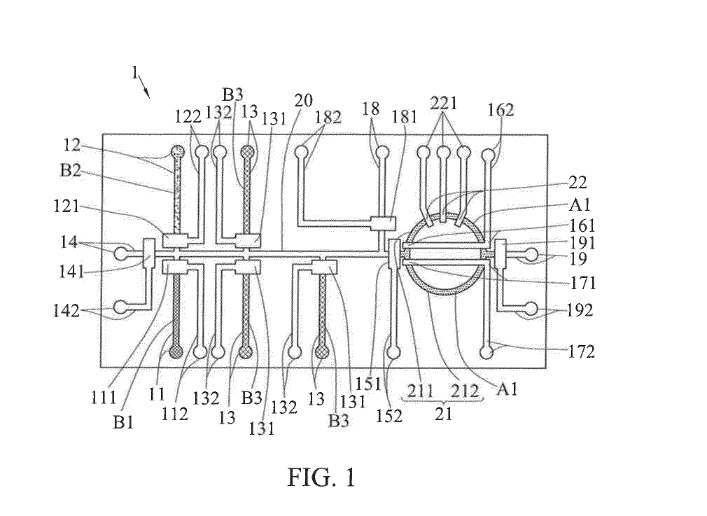

FIG. 1 is a top view of a microfluidic chip 1 for culturing microorganisms according to the present invention. In an embodiment, the microfluidic chip 1 has a nano-level size, a micro capacity and a micro volume, and consumes low power. The microfluidic chip 1 comprises a first input unit 11, a second input unit 12, a connection unit 20, a ring-shaped storage structure 21, and a plurality of control valves 22.

The first input unit 11 provides a first solution B1. The second input unit 12 provides a second solution 132. The connection unit 20 is a communication channel that is connected to the first input unit 11 and the second input unit 12.

The ring-shaped storage structure 21 is connected to the connection unit 20, and has a first growth chamber 211 and a second growth chamber 212 that store a microbial solution A1. The first growth chamber 211 stores a half of the microbial solution A1, and the second growth chamber 212 stores the other half. In an embodiment, the microbial solution A1 is a bacteria solution for growing bacteria, or a cell solution for growing cells.

The control valves 22 are disposed at one or both of the first growth chamber 211 and the second growth chamber 212. The control valves 22, after actuated, mix the second solution 132 and remainder of microbial solution A1 in the ring-shaped storage structure 21.

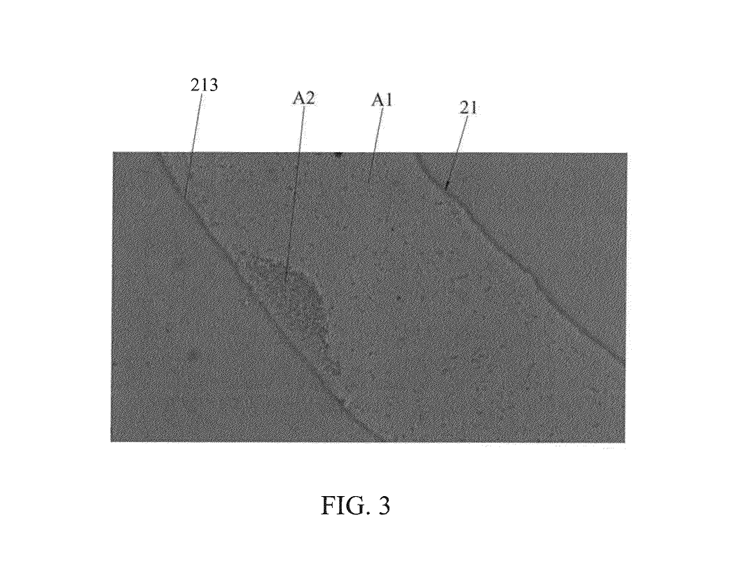

In an embodiment, the first solution B1 is a lysis solution or a cleaning solution, and is transferred through the connection unit 20 into the first growth chamber 211 or the second growth chamber 212 to discharge the stored microbial solution A1 to an external region. In an embodiment, the first solution B1 lysis or cleans a biological membrane (see FIG. 3) and planktons adhered to a pipe wall of the ring-shaped storage structure 21 and produced by the microbial solution A1.

In an embodiment, the second solution B2 is a nutrient solution or a buffer solution, and is transferred through the connection unit 20 into the first growth chamber 211 or the second growth chamber 212 to discharge the first solution B1 to an external region. In an embodiment, the second solution B2 provides nutrients to microorganisms in the microbial solution A1, and is mixed with the microbial solution A1 N times such that the microbial solution A1 is diluted and has a concentration equal to (½)^{N} of its original concentration, where n is a positive integer.

The microfluidic chip 1 includes a first valve 111 disposed between the first input unit 11 and the connection unit 20, a first control unit 112 connected to and controlling (e.g., opening or closing) the first valve 111, a second valve 121 disposed between the second input unit 12 and the connection unit 20, and a second control unit 122 connected to and controlling (e.g., opening or closing) the second valve 121.

The microfluidic chip 1 comprises a mixing unit 221 that mixes the second solution B2 and the remainder of the microbial solution A1 in the ring-shaped storage structure 21 according to a predetermined rotation direction (e.g., clockwise or counter-clockwise).

The microfluidic chip 1 comprises at least one third input unit 13, at least one third valve 131, and at least one third control unit 132. The third input unit 13 provides at least one third solution B3. The third valve 131 is disposed between the third input unit 13 and the connection unit 20. The third control unit 132 is connected to and controls (e.g., opening or closing) the third valve 131.

In an embodiment, the microfluidic chip 1 includes three third input units 13, three third valves 131, and three third control units 132. The three third input units 13 provide three different third solutions B3 (e.g., an antibiotic solution), allowing a user to obtain a variety of drug reactions induced from the reactions of the three third solutions B3 with the microorganisms in the microbial solution A1.

The microfluidic chip 1 comprises a gas input unit 14 that is connected to the connection unit 20 and inputs gas into the connection unit 20, a fourth valve 141 disposed between the gas input unit 14 and the connection unit 20, and a fourth control unit 142 connected yo and controlling (e.g., opening or closing) the fourth valve 141.

The microfluidic chip 1 comprises a fifth valve 151 disposed between the connection unit 20 and the ring-shaped storage structure 21, and a fifth control unit 152 connected to and controlling (e.g., opening or closing) the fifth valve 151.

The microfluidic chip 1 comprises two sixth valves 161, a sixth control unit 162, two seventh valves 171, and a seventh control unit 172. The two sixth valves 161 are disposed on two ends of the first growth chamber 211, respectively. The control valves 22 are disposed between the two sixth valves 161. The sixth control unit 162 is connected to and controls (e.g., opening or closing) the two sixth valves 161. The two seventh valves 171 are disposed on two ends of the second growth chamber 212, respectively. The seventh control unit 172 is connected to and controls (e.g., opening or closing) the two seventh valves 171.

The microfluidic chip 1 comprises a first output unit 18, an eighth valve 181, and an eighth control unit 182. The first output unit 18 is connected to the connection unit 20. The eighth valve 181 is disposed between the first output unit 18 and the connection unit 20. The eighth control unit 182 is connected to and controls (e.g., opening or closing) the eighth valve 181.

The microfluidic chip 1 comprises a second output unit 19, a ninth valve 191, and a ninth control unit 192. The second output unit 19 is connected to the ring-shaped storage structure 21. The ninth valve 191 is disposed between the ring-shaped storage structure 21 and the second output unit 19. The ninth control unit 192 is connected to and controls (e.g., opening or closing) the ninth valve 191.

In an embodiment, each of the mixing unit 221 and the first control unit 112 to the ninth control unit 192 comprises pores and communication channels. The pores are connected to a gas supply device (not shown) such as a pump that provides gas. The communication channels have fluid filled therein. The gas flowing into or out of the pores applies a positive pressure or a negative pressure to the fluid in the communication channels to control (e.g., opening or closing) the control valves 22 and the first valve 111 to the ninth valve 191.

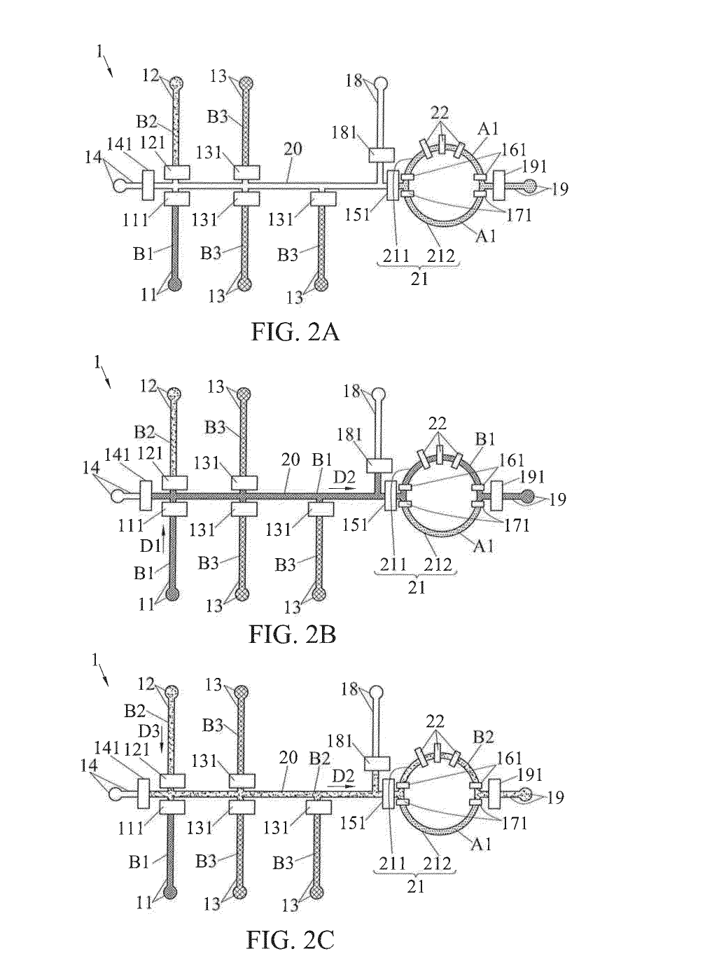

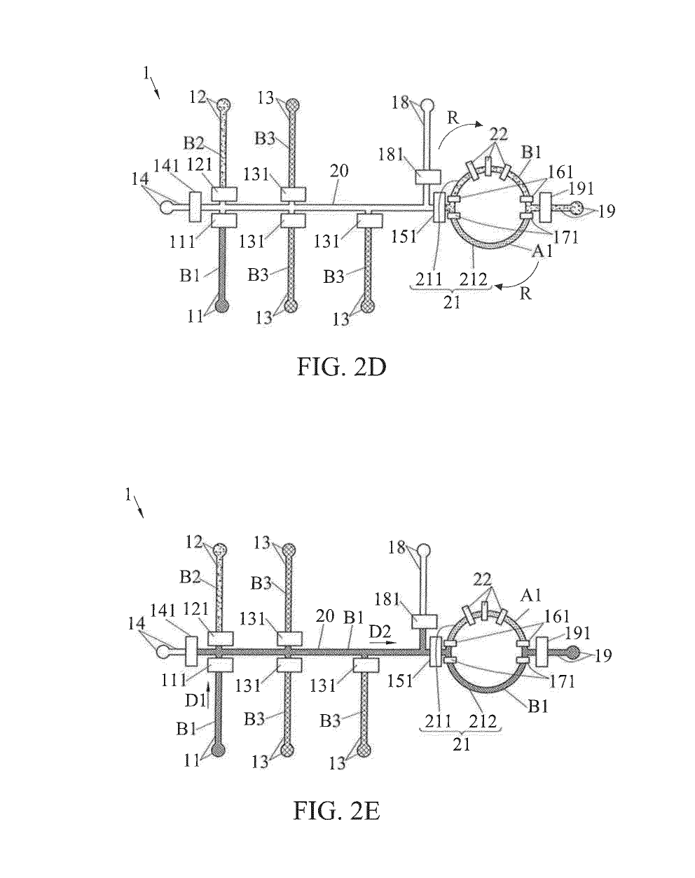

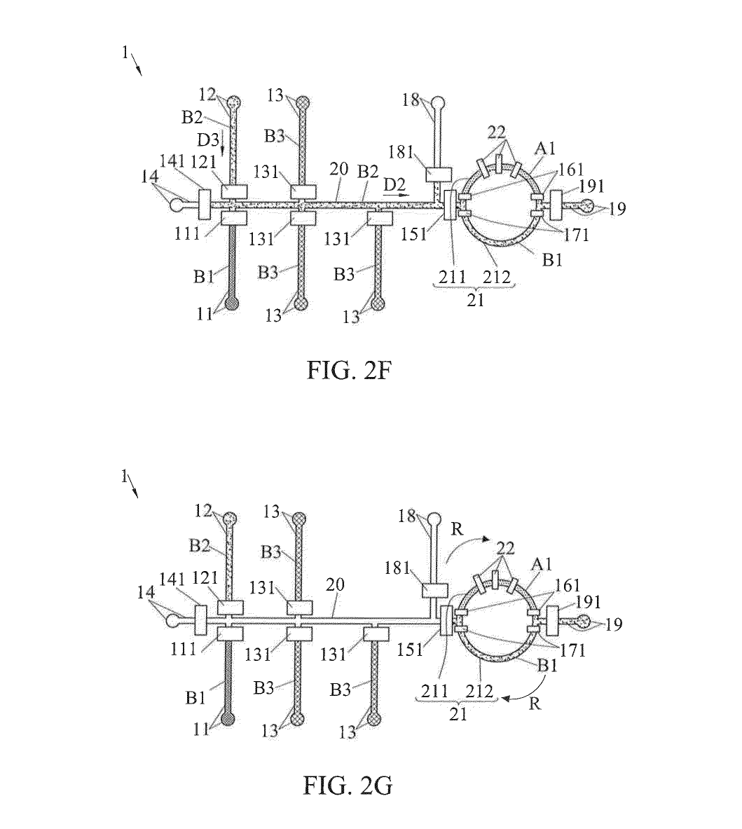

FIGS. 2A to 2G are top views illustrating a method for operating a microfluidic chip 1 for culturing microorganisms according to the present invention. The mixing unit 221 and the first control unit 112 to the ninth control unit 192 are not shown in FIGS. 2A to 2G, in order to highlight the operations of the remaining components of the microfluidic chip 1 and the first solution 131 to the third solution B3.

As shown in FIG. 2A and FIG. 1, a microfluidic chip 1 is provided that has a first input unit 11, a second input unit 12, a connection unit 20, a ring-shaped storage structure 21, and a plurality of control valves 22.

The connection unit 20 is connected to the first input unit 11 and the second input unit 12. The ring-shaped storage structure 21 is connected to the connection unit 20, and has a first growth chamber 211 and a second growth chamber 212 that store a microbial solution A1. The first growth chamber 211 stores a half of the microbial solution A1, and the second growth chamber 212 stores the other half. The control valves 22 are disposed at the first growth chamber 211.

The microfluidic chip 1 further has the mixing unit 221, the gas input unit 14, the first valve 111 to the ninth valve 191, and the first control unit 112 to the ninth control unit 192, further description thereof hereby omitted.

As shown in FIG. 2B and FIG. 1, the first control unit 112, the fifth control unit 152, the sixth control unit 162 and the ninth control unit 192 of the microfluidic chip 1 control the first valve 111, the fifth valve 151, the two sixth valves 161 and the ninth valve 191, respectively, to input the first solution B1 from the first input unit 11 according to a predetermined direction D1 and transfer the first solution B1 through the connection unit 20 into the first growth chamber 211 according to a predetermined direction D2 to treat and discharge the microbial solution A1 in the first growth chamber 211 to a region outside of the second output unit 19.

In an embodiment, the first solution B1 is a lysis solution or a cleaning solution that lysis or cleans a biological membrane (see FIG. 3) and planktons adhered to a pipe wall of the ring-shaped storage structure 21 and produced by the microbial solution A1.

As shown in FIG. 2C and FIG. 1, the second control unit 122, the fifth control unit 152, the sixth control unit 162 and the ninth control unit 192 of the microfluidic chip 1 control the second valve 121, the fifth valve 151, the two sixth valves 161 and the ninth valve 191, respectively, to input the second solution B2 from the second input unit 12 according to a predetermined direction D3 and transfer the second solution B2 through the connection unit 20 into the first growth chamber 211 according to a predetermined direction D2, to discharge the first solution B1 in the first growth chamber 211 to a region outside of the second output unit 19.

As shown in FIG. 2D and FIG. 1, the mixing unit 221 of the microfluidic chip 1 actuates the control valves 22 to mix the second solution B2 and the remainder of the microbial solution A1 in the ring-shaped storage structure 21 according to a predetermined rotation direction R (e.g., a clockwise direction).

In an embodiment, the second solution B2 is a nutrient solution or a buffer solution that provides nutrients to the microorganisms in the microbial solution A1, and the microbial solution A1 is diluted by the second solution B2 and has a concentration equal to a half of a concentration of the microbial solution A1 shown in FIG. 2A.

The fourth control unit 142 and the eighth control unit 182 of the microfluidic chip 1 control the fourth valve 141 and the eighth valve 181, respectively, to input gas from the gas input unit 14 of the microfluidic chip 1 into the connection unit 20 and discharge the second solution B2 in the connection unit 20 through the first output unit 18 to an external region.

As shown in FIG. 2E and FIG. 1, the first control unit 112, the fifth control unit 152, the seventh control unit 172 and the ninth control unit 192 of the microfluidic chip 1 control the first valve 111, the fifth valve 151, the two seventh valves 171 and the ninth valve 191 to input the first solution B1 from the first input unit 11 according to a predetermined direction D1 and transfer the first solution B1 through the connection unit 20 into the second growth chamber 212 according to a predetermined direction D2 to treat and discharge the microbial solution A1 in the second growth chamber 212 to a region outside of the second output unit 19.

As shown in FIG. 2F and FIG. 1, the second control unit 122, the fifth control unit 152, the seventh control unit 172 and the ninth control unit 192 of the microfluidic chip 1 control the second valve 121, the fifth valve 151, the two seventh valves 171 and the ninth valve 191, respectively, to input the second solution B2 from the second input unit 12 according to a predetermined direction D3 and transfer the second solution 132 through the connection unit 20 into the second growth chamber 212 according to a predetermined direction D2 to discharge the first solution 131 in the second growth chamber 212 to a region outside of the second output unit 19.

As shown in FIG. 2G and FIG. 1, the mixing unit 221 of the microfluidic chip 1 actuates the control valves 22 and mix the second solution B2 and the remainder of the microbial solution A1 in the ring-shaped storage structure 21 according to a predetermined rotation direction R (e.g., a clockwise direction) to dilute the microbial solution A1 such that the concentration of the diluted microbial solution A1 is a half of the concentration of the microbial solution A1 shown in FIG. 2D, and is a quarter of a concentration of the microbial solution A1 shown in FIG. 2A.

The fourth control unit 142 and the eighth control unit 182 of the microfluidic chip 1 control the fourth valve 141 and the eighth valve 181, respectively, to input gas from the gas input unit 14 of the microfluidic chip 1 into the connection unit 20 to discharge the second solution B2 in the connection unit 20 through the first output unit 18 to an external region.

In addition to the processes of FIGS. 2A-2G, at least one third input unit 13 of the microfluidic chip 1 provides at least one third solution B3, and at least one third control unit 132 of the microfluidic chip 1 controls at least one third valve 131 to input the third solution B3 from the third input unit 13.

In an embodiment, three third input units 13 provide three different third solutions B3 (e.g., an antibiotic solution), and three third control units 132 control the three third valves 131, respectively, to input at least one of the three third solutions B3 from the three third input units 13, allowing a user to obtain a variety of drug reactions induced from the reactions of the three third solutions B3 with the microorganisms in the microbial solution A1.

FIG. 3 is an image of a biological membrane adhered to a pipe wall 213 of a ring-shaped storage structure 21 according to the present invention.

The pipe wall 213 of the ring-shaped storage structure 21 is adhered with a biological membrane A2 and/or planktons that are produced by the microbial solution A1.

In an embodiment, the processes shown in Fiefs. 2A-2G are performed such that the first solution B1 (e.g., a lysis solution or a cleaning solution) lysis or removes the biological membrane A2 on the pipe walls 213 of the first growth chamber 211 and the second growth chamber 212.

In another embodiment, only the processes shown in FIGS. 2A-2D are performed such that the first solution B1 lysis and removes the biological membrane A2 on the pipe wall 213 of the first growth chamber 211, without lysising or removing the biological membrane A2 on the pipe wall 213 of the second growth chamber 212. Therefore, a user is allowed to obtain and observe the variation of the biological membrane A2.

In yet another embodiment, only the processes shown in FIGS. 2A and 2E-2G are performed such that the first solution B1 lysis and removes the biological membrane A2 on the pipe wall 213 of the first growth chamber 211, without lysising and removing the biological membrane A2 on the pipe wall 213 of the second growth chamber 212. Therefore, a user is allowed to obtain and observe the variation of the biological membrane A2.

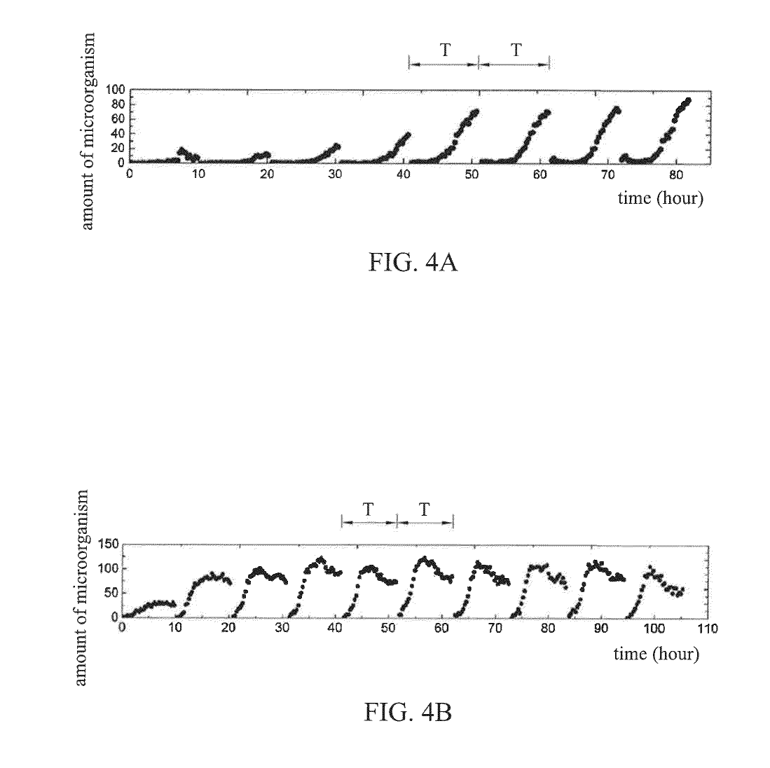

FIG. 4A is a growth curve of an amount of micro microorganisms obtained by a microfluidic chip 1 for culturing microorganisms and a method of operating the microfluidic chip 1 according to the present invention, which includes a result obtained from performing the processes shown in FIGS. 2A-2G iteratively.

At a time point P1 during each period T (e.g., about 10.5 hours), an amount of the microorganisms in the ring-shaped storage structure 21 is decreased and equal to a quarter thereof or within a predetermined range such that a growth amount or a growth curve of the microorganisms is controlled.

In FIG. 4A, since only a portion of the microorganisms in the ring-shaped storage structure 21 is observed, and some of the microorganisms are adhered to the pipe wall of the ring-shaped storage structure 21, less than a quarter of the microorganisms are shown in FIG. 4A.

FIG. 4B is another growth curve of an amount of micro microorganisms obtained by a microfluidic chip for culturing microorganisms and a method of operating the microfluidic chip according to the present invention, which includes a result obtained from performing the processes shown in FIGS. 2A-2G iteratively.

FIGS. 4A and 4B are different in that FIG. 4A employs a lysis solution, while FIG. 4B employs a deionized water (DI) cleaning solution.

At a time point P1 during each period T (e.g., about 10.5 hours), an amount of the microorganisms in the ring-shaped storage structure 21 is decreased and equal to a quarter thereof or within a predetermined range such that a growth amount or a growth curve of the microorganisms is controlled.

Since the pipe wall 213 of the ring-shaped storage structure 21 is adhered with the biological membrane A2 shown in FIG. 3, and the biological membrane A2 absorbs the microorganisms in the microbial solution A1, the amount of the microorganism in the microbial solution A1 at a time point P2 during each period T is decreased.

In a microfluidic chip for culturing microorganisms and a method of operating the same according to the present invention, a first input unit, a second input unit and a third input unit provide a first solution (e.g., a lysis solution), a second solution (e.g., a nutrient solution) and a third solution (e.g., an antibiotic solution), respectively, and transfer the first to third solutions to the first or the second growth chamber of the ring-shaped storage structure, to lysis a biological membrane, mix (dilute) the microbial solution and control the growth of microorganisms.

Therefore, the present invention removes a biological membrane on the pipe wall of the ring-shaped storage structure, and dilutes the microbial solution such that the microbial solution has a concentration equal to (½)^{N} of its original concentration after n periods. Therefore, a growth amount of the microorganisms is controlled to be within a predetermined range and stable, and the drug reactions of the microorganisms with antibiotics can also be obtained.

A microfluidic chip according to the present invention has a nano-level size, a micro capacity and a micro volume, and consumes less nutrient solution. Compared with the prior art, the microfluidic chip according to the present invention has a more simplified structure and a lower cost.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. A microfluidic chip for culturing microorganisms, comprising:
a first input unit that provides a first solution;
a second input unit that provides a second solution;
a connection unit connected to the first input unit and the second input unit;
a ring-shaped storage structure connected to the connection unit and having a first growth chamber and a second growth chamber that store a microbial solution; and
a plurality of control valves disposed at the first growth chamber,
wherein the first solution is transferred through the connection unit into the first growth chamber or the second growth chamber to treat and discharge a portion of the microbial solution, the second solution is transferred through the connection unit into the first growth chamber or the second growth chamber to discharge the first solution, and the control valves, after actuated, mix the second solution and remainder of the microbial solution in the ring-shaped storage structure.

2. The microfluidic chip of claim 1, further comprising a first valve disposed between the first input unit and the connection unit, a first control unit connected to the first valve, a second valve disposed between the second input unit and the connection unit, and a second control unit connected to the second valve.

3. The microfluidic chip of claim 1, further comprising a mixing unit connected to the control valves to actuate the control valves and mix the second solution and the remainder of the microbial solution in the ring-shaped storage structure according to a predetermined rotation direction.

4. The microfluidic chip of claim 1, further comprising at least one third input unit, at least one third valve, and at least one third control unit, wherein the third input unit provides at least one third solution, the third valve is disposed between the third input unit and the connection unit, and the third control unit is connected to the third valve.

5. The microfluidic chip of claim 4, wherein the first solution is a lysis solution or a cleaning solution, the second solution is a nutrient solution or a buffer solution, and the third solution is an antibiotic solution.

6. The microfluidic chip of claim 1, further comprising a gas input unit connected to the connection unit, a fourth valve disposed between the gas input unit and the connection unit, and a fourth control unit connected to the fourth valve.

7. The microfluidic chip of claim 1, further comprising a fifth valve disposed between the connection unit and the ring-shaped storage structure, and a fifth control unit connected to the fifth valve.

8. The microfluidic chip of claim 1, further comprising two sixth valves, a sixth control unit, two seventh valves, and a seventh control unit, wherein the two sixth valves are disposed on two ends of the first growth chamber, respectively; the control valves are disposed between the two sixth valves, the sixth control unit is connected to the two sixth valves, the two seventh valves are disposed on two ends of the second growth chamber, respectively; and the seventh control unit is connected to the two seventh valves.

9. A method of operating a microfluidic chip for culturing microorganisms, the method comprising:
providing a microfluidic chip that has a first input unit, a second input unit, a connection unit, a ring-shaped storage structure, and a plurality of control valves, wherein the connection unit is connected to the first input unit and the second input unit, the ring-shaped storage structure is connected to the connection unit, the ring-shaped storage structure has a first growth chamber and a second growth chamber that store a microbial solution, and the control valves are disposed at the first growth chamber;
inputting a first solution from the first input unit and transferring the first solution through the connection unit into the first growth chamber or the second growth chamber to treat and discharge a portion of the microbial solution;
inputting a second solution from the second input unit and transferring the second solution through the connection unit into the first growth chamber or the second growth chamber to discharge the first solution; and
actuating the control valves to mix the second solution and remainder of the microbial solution in the ring-shaped storage structure.

10. The method of claim 9, further comprising:
controlling a first valve by a first control unit of the microfluidic chip to input the first solution from the first input unit; and
controlling a second valve by a second control unit of the microfluidic chip to input the second solution from the second input unit.

11. The method of claim 9, further comprising actuating the control valves by a mixing unit of the microfluidic chip to mix the second solution and the remainder of the microbial solution in the ring-shaped storage structure according to a predetermined rotation direction.

12. The method of claim 9, further comprising:
providing at least one third solution by at least one third input unit of the microfluidic chip; and
controlling at least one third valve by at least one third control unit of the microfluidic chip to input the third solution from the third input unit.

13. The method of claim 12, wherein the first solution is a lysis solution or a cleaning solution, the second solution is a nutrient solution or a buffer solution, and the third solution is an antibiotic solution.

14. The method of claim 9, further comprising controlling a fourth valve by a fourth control unit of the microfluidic chip to input gas into the connection unit by a gas input unit of the microfluidic chip.

15. The method of claim 9, further comprising controlling a fifth valve by a fifth control unit of the microfluidic chip to transfer the first solution or the second solution through the connection unit to the ring-shaped storage structure.

16. The method of claim 9, further comprising:
controlling two sixth valves by a sixth control unit of the microfluidic chip to transfer the first solution or the second solution into the first growth chamber; and
controlling two seventh valves by a seventh control unit of the microfluidic chip to transfer the first solution or the second solution into the second growth chamber.
